(19) United States
(12) Patent Application Publication
(10) Pub. No.: US 2010/0145454 A1
(43) Pub. Date: Jun. 10, 2010

(54) INTERVERTEBRAL DISC NUCLEUS REPLACEMENT PROSTHESIS

(75) Inventor: Zachary M. Hoffman, Bloomington, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: Zimmer Spine, Inc., Minneapolis, MN (US)

(21) Appl. No.: 12/331,019

(22) Filed: Dec. 9, 2008

(51) Int. Cl. A61F 2/44

(52) U.S. Cl. 623/17.12; 623/17.11; 623/17.16

(57) ABSTRACT

A nucleus replacement prosthesis for a nucleus of an intervertebral disc. The nucleus replacement prosthesis includes an elastic porous construct having a plurality of interconnected pores. The porous construct may be formed of a non-hydrogel material. The porous construct is compressible by the application of an applied compressive force from an enlarged state to a smaller compressed state. The porous construct is predisposed to assume the enlarged state in the absence of the applied compressive force without hydration of the porous construct. A substance may be located in the interconnected pores of the porous construct.

### TECHNICAL FIELD

The disclosure is directed to a prosthesis for an intervertebral disc, more specifically a nucleus replacement prosthesis. More particularly, the disclosure is directed to a construct which can be placed in a nuclear void established in an intervertebral disc.

### BACKGROUND

The human spinal column includes intervertebral discs positioned between adjacent vertebrae. The intervertebral discs distribute forces between adjacent vertebrae while stabilizing the spinal column. Intervertebral discs are composed of three regions; the nucleus pulposus, the annulus fibrosus, and the vertebral end plates. The nucleus pulposus, which has a high proteoglycan content, contains about 70-90% water, giving the nucleus pulposus a gelatin-like consistency. The annulus fibrosus, formed of fibrous tissue, surrounds the nucleus pulposus, providing hoop strength and support to the nucleus pulposus when subjected to compressive loading.

Degeneration, displacement or damage of the intervertebral disc may lead to instability of the spine, decreased mobility, nerve damage and/or pain. Therefore, there is an ongoing desire to provide prosthetic implants to replace the damaged portion of the intervertebral disc, such as the nucleus pulposus of the intervertebral disc, in order to restore stability/mobility of the spine, provide height restoration, and/or reduce/eliminate pain.

### SUMMARY

The disclosure is directed to several alternative designs, materials, manufacturing processes and methods of use of medical device structures and assemblies.

Accordingly, one illustrative embodiment is a nucleus replacement prosthesis for a nucleus of an intervertebral disc. The nucleus replacement prosthesis includes an elastic porous construct formed from a non-hydrogel material. The porous construct, which may resemble a sponge, includes a plurality of interconnected pores and is compressible by the application of an applied compressive force from an enlarged state to a smaller compressed state. The elastic porous construct is predisposed (e.g., biased) to assume the enlarged state in the absence of the applied compressive force. A substance may fill or substantially fill the interconnected pores of the elastic porous construct.

Another illustrative embodiment is a nucleus replacement prosthesis for a nucleus of an intervertebral disc. The nucleus replacement prosthesis includes an elastic porous construct having a plurality of interconnected pores. The elastic porous construct is compressible by the application of an applied compressive force from an enlarged state to a smaller compressed state for insertion into a nuclear void of the intervertebral disc. The elastic porous construct is predisposed (e.g., biased) to assume the enlarged state in the absence of the applied compressive force without hydration of the elastic porous construct such that the elastic porous construct may substantially fill the nuclear void. A substance may be located in the interconnected pores of the elastic porous construct to provide the nucleus replacement prosthesis with support to withstand loading of the intervertebral disc by the spinal column.

Yet another illustrative embodiment is a method of replacing a nucleus of an intervertebral disc. The method includes removing at least a portion of the nucleus to create a nuclear void within an annulus fibrosus of the intervertebral disc. A porous construct is then inserted in a compressed state into the nuclear void. The porous construct is expanded within the nuclear void to an expanded state. Once expanded, a substance is injected into the expanded porous construct such that the substance fills a plurality of interconnected pores of the porous construct.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a perspective view of an illustrative nucleus replacement prosthesis;

FIG. 2A is a cross-sectional view of the nucleus replacement prosthesis of FIG. 1 taken through line 2-2;

FIG. 2B is an alternative cross-sectional view of the nucleus replacement prosthesis of FIG. 1 taken through line 2-2;

FIG. 2C is another alternative cross-sectional view of the nucleus replacement prosthesis of FIG. 1 taken through line 2-2;

FIG. 3 is an enlarged view of a portion of the porous construct of the nucleus replacement prosthesis of FIG. 1;

FIGS. 4-9 illustrate one exemplary procedure of installing a nucleus replacement prosthesis in a nuclear void of an intervertebral disc; and

FIG. 10 is a partially cut-away view of another nucleus replacement prosthesis.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the term “hydrogel” refers to a class of crosslinked polymeric materials which have an affinity to absorb water, and typically swell when bonded to water, through the formation of hydrogen bonds between the crosslinked polymeric material and H₂0.

As used in this specification and the appended claims, the term “non-hydrogel” refers to a class of materials which are not classified as a hydrogel.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

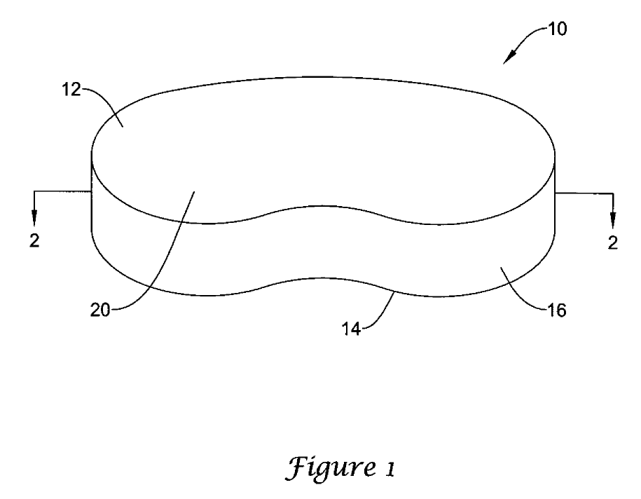

Referring to FIG. 1, there is illustrated a prosthetic implant for an intervertebral disc, shown as a nucleus replacement prosthesis 10 to replace a displaced, diseased degenerated or damaged nucleus pulposus of an intervertebral disc. The nucleus replacement prosthesis 10 may include an upper surface 12, a lower surface 14 spaced from the upper surface 12, and a peripheral surface 16 extending between the upper surface 12 and the lower surface 14. The nucleus replacement prosthesis 10 may have a shape and/or size similar to the native nucleus pulposus of an intervertebral disc. As shown in FIG. 1, in some embodiments, the nucleus replacement prosthesis 10 may be kidney shaped, while in other embodiments the nucleus replacement prosthesis 10 may be disc shaped, cylindrical, ovoid, annular, rectangular, or other desired shape to fill a nuclear void. In some embodiments, the nucleus replacement prosthesis 10 may be egg shaped, having convex upper and lower surfaces, as well as convex side surfaces extending around a periphery of the nucleus replacement prosthesis 10.

The nucleus replacement prosthesis 10 may include an elastic, porous construct 20 including a plurality of interconnected pores. For example, the porous construct 20 may be a sponge-like porous scaffold having a plurality of interconnected pores located throughout the porous scaffold. In some embodiments the porous construct 20 may be a highly open porous structure with well interconnected pores. In some embodiments the porous construct 20 may have a porosity (e.g., the percentage of interstitial volume (pores) to total volume of the porous construct) of 50% or more, 60% or more, 75% or more, 85% or more, or 90% or more. The porosity and/or average pore size may provide the porous construct 20 with desired functional attributes desired to replicate a native nucleus pulposus.

The porous construct 20 may be formed of a variety of materials. For example, the porous construct 20 may be formed of a non-hydrogel material. For instance, the porous construct 20 may be formed of an uncrosslinked polymer material, a polymeric foam material, such as an open cell polymeric foam material, or other polymeric materials. In some embodiments, the porous construct 20 may be formed of a urethane material, such as a urethane foam material. Other possible materials for the porous construct 20 include porous metals and metallic materials, porous composite materials, as well as other porous materials which are compressible in a porous state.

In some embodiments, the porous construct 20 may be formed of a non-hydrophilic polymer, or other polymer material, having surface-modifying endgroups (SME) and/or surface-modifying macromolecules (SMM), creating hydrophilic surfaces on the construct 20 formed of a non-hydrophilic polymer. For instance, a porous construct 20 which includes surface-modifying endgroups and/or surface-modifying macromolecules may result in the surfaces of the walls of the porous construct 20 having an affinity for interdiscal fluid, water and/or other fluid, drawing the interdiscal fluid, water and/or other fluid into the pores of the porous construct 20. Surface-modifying endgroups are surface-active oligomers covalently bonded to the base polymer during synthesis. Some surface-modifying endgroups include silicone, sulfonate, fluorocarbon, polyethylene oxide, and hydrocarbon chains. Surface-modifying macromolecules are oligomeric fluoropolymers synthesized by polyurethane chemistry and tailored with fluorinated end groups.

The material for the porous construct 20 may be chosen to be elastic such that the porous construct 20, at least in the absence of a material occupying the pores of the porous construct 20, is compressible by the application of an applied compressive force from an enlarged state to a smaller compressed state. In some embodiments, the porous construct 20 may be compressed to one-fifth or less, one-tenth or less, one-twentieth or less, one-thirtieth or less, or one-fiftieth or less of its size in the expanded state. Resultant of the elastic nature of the porous construct 20, the porous construct 20 is predisposed (e.g., biased) to assume the enlarged state in the absence of the applied compressive force, without the need to be hydrated. Thus, due to the elastic nature of the porous construct 20, the porous construct 20 will automatically expand when not subjected to a constraining and/or compressive force. Unlike a hydrogel, the porous construct 20 is expandable in situ without requiring hydration of the porous construct 20.

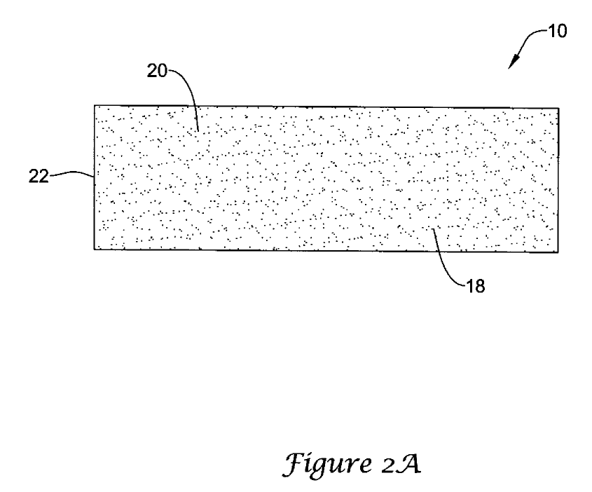

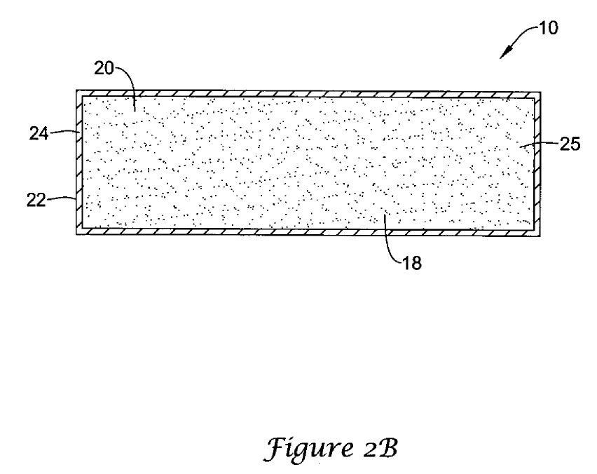

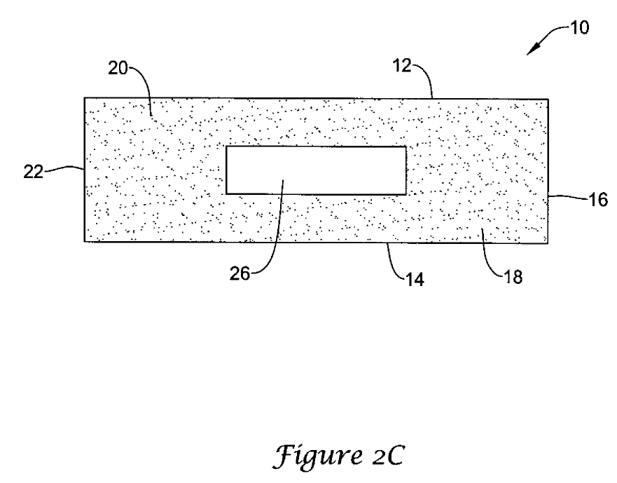

FIG. 2A is a cross-section taken of the nucleus replacement prosthesis 10 of FIG. 1. As shown in FIG. 2A, in some embodiments, the porous nature, e.g., porosity, of the porous construct 20 may extend throughout the interior 18 of the porous construct 20. Additionally, the porous nature, e.g., porosity, of the porous construct 20 may extend to the exterior surface 22 of the porous construct 20 such that the exterior surface 22 of the porous construct 20 has an open porosity in which pores at the exterior surface 22 are interconnected with pores in the interior 18 of the porous construct 20.

FIG. 2B is an alternate cross-section taken of the nucleus replacement prosthesis 10 of FIG. 1. As shown in FIG. 2B, in some embodiments, the porous nature, e.g., porosity, of the porous construct 20 may extend throughout the interior 18 of the porous construct 20. Furthermore, as shown in FIG. 2B, the exterior surface 22 of the porous construct 20 may include a containing component 24 surrounding an interior core portion 25 of the porous construct 20. In some embodiments, the containing component 24 may be porous or non-porous and/or permeable or non-permeable. In some embodiments, the containing component 24 may limit expansion of the interior core portion 25 of the porous construct 20. In some embodiments, the containing component 24 may be formed of a dissimilar material from that of the interior core portion 25 of the porous construct 20. In other embodiments, the containing component 24 may be formed of the same material as that of the interior core portion 25 of the porous construct 20. In some embodiments, the containing component 24 may be a thickness of the porous construct 20 in which the pores of the porous construct 20 exposed to the exterior surface 22 are filled, reformed, closed, or otherwise altered from the configuration of pores of the interior core portion 25 of the porous construct 20. In some embodiments, the containing component 24 of the porous construct 20 may have a rigidity greater than the rigidity of the interior core portion 25 of the porous construct 20. In some embodiments, the presence of the containing component 24, surrounding the interior core portion 25 of the porous construct 20 may retain a substance, such as a fluid, gel, or other material within the pores of the interior core portion 25 of the porous construct 20.

FIG. 2C is another alternate cross-section taken of the nucleus replacement prosthesis 10 of FIG. 1. As shown in FIG. 2C, in some embodiments, the interior 18 of the porous construct 20 may have a porous nature, e.g., porosity. Additionally, the porous nature, e.g., porosity, of the porous construct 20 may extend to the exterior surface 22 of the porous construct 20 such that the exterior surface 22 of the porous construct 20 has an open porosity in which pores at the exterior surface 22 are interconnected with pores in the interior 18 of the porous construct 20. Furthermore, as shown in FIG. 2C, in some embodiments, the porous construct 20 may include one or more cavities 26 located in the porous construct 20. A single cavity 26 is illustrated in FIG. 2C, however, in other embodiments, the porous construct 20 may include two, three, four, five, six, or more cavities as desired. The cavity 26 may be magnitudes larger than the individual pores of the porous construct 20, thus the cavity 26 is to be distinguished from the pores of the porous construct 20. In some embodiments, the cavity 26 may be located within the porous construct 20 such that no portion of the cavity 26 extends to an exterior surface 22 of the porous construct 20, such as the upper surface 12, the lower surface 14 and/or the peripheral surface 16 of the porous construct 20. In other embodiments, the cavity 26 may extend to one or more of the upper surface 12, the lower surface 14 and/or the peripheral surface 16 of the porous construct 20. In some embodiments, the cavity 26 may act as a reservoir for holding a fluid. During spinal loading, fluid in the cavity 26 may be pushed out into the pores of the porous construct 20, and during spinal offloading, fluid may flow back into the cavity 26 from the pores of the porous construct 20. Such movement of fluid into and out of the pores of the porous construct 20 may replicate diurnal pumping of a native nucleus pulposus of an intervertebral disc.

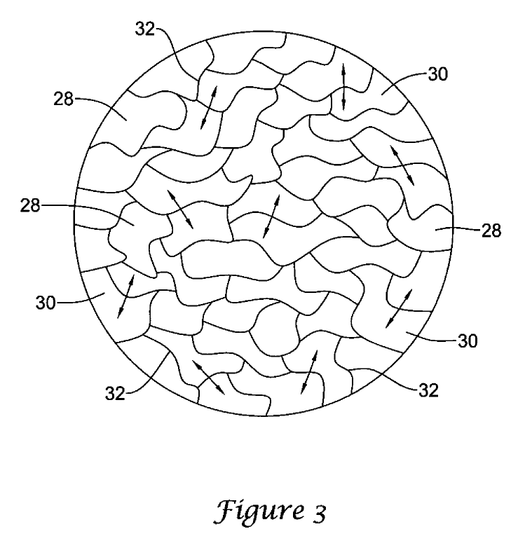

FIG. 3 is an enlarged view of a portion of the porous construct 20 of the nucleus replacement prosthesis 10 illustrating the porous nature, e.g., porosity, of the porous construct 20. As shown in FIG. 3, the porous construct 20 may be a cellular matrix of a plurality of interconnected pores 28. A plurality of interconnected walls 32 of the cellular matrix of the porous construct 20 define the pores 28. As will be discussed further herein, once the porous construct 20 is expanded to fill a nuclear void of an intervertebral disc, a substance 30, such as a filler material, a fluid, or other material, may be introduced into the pores 28 of the porous construct 20. The substance 30 introduced into the pores 28 may provide the nucleus replacement prosthesis 10 with sufficient structure to carry loads experienced by an intervertebral disc in conjunction with the annulus fibrosus. The porous construct 20, alone may be insufficient for carrying such loads.

In some embodiments, the substance 30 occupying the pores 28 may be chosen to provide the nucleus replacement prosthesis 10 with a degree of dynamic loading/offloading capabilities. For example, the substance 30 may be a polymer, fluid (including gels), bone graft putty, or any flowable material including flowable solids such as small particles, polymer or ceramic beads, or a material capable of curing in situ. In some embodiments, the substance 30 may be silicone, saline, urethane, gel, water, or other desired substance. In some embodiments, the substance 30 may be less stiff than the material forming the cellular matrix of the porous construct 20, while in other embodiments the substance 30 may be more stiff than the material forming the cellular matrix of the porous construct 20. As depicted by the arrows of FIG. 3, in some embodiments the substance 30 may flow between adjacent interconnected pores 28 of the porous construct 20 through cyclic loading/offloading of the nucleus replacement prosthesis 10, resembling the diurnal pumping action associated with a native nucleus pulposus of an intervertebral disc. The movement of the substance 30 into and out of pores 28 of the porous construct 20 may create a poroelastic construct. Behavior of a poroelastic construct is dependent on the deformation of the cellular matrix of the porous construct 20 and the movement of the substance 30 (e.g., fluid) in and out of pores of the porous construct 20 during deformation, resulting in time dependent stress-strain behavior (e.g., stress relaxation and creep) of the nucleus replacement prosthesis 10.

In embodiments in which the substance 30 is a fluid, the porous construct 20 may have an affinity (i.e., an attractive force between substances tending to cause the substances to enter into and remain in chemical combination) to attract fluid, such as water, saline and/or interdiscal fluid. In some embodiments, the affinity may be from chemical bonding, such as covalent bonding or hydrogen bonding, between the substance 30 and the porous construct 20, for example.

In other embodiments, the substance 30 may be an in situ curing material which may be injected into the pores of the porous construct 20 once the porous construct 20 has expanded in the nuclear void and then allowed to cure. Exemplary in situ curable polymers include bone cement, polyurethanes or other in situ curable elastomers or polymers. The substance 30 may be capable of being cured to a semi-rigid or rigid state capable of supporting loading of the spinal column. In some embodiments, the substance 30 may be chosen to be self-hardening or self-curable, or hardenable or curable upon the application of heat, light, air, a curing agent or other hardening or curing means. In some instances the substance 30 may be an ultraviolet (UV) curable material, including UV curable silicones, urethanes, and other polymers. In other instances the substance 30 may be a thermally curable material, including thermally curable polymers. The substance 30 may be chosen to harden or cure shortly after injection into the pores 28 or over a period of hours or days, in some instances.

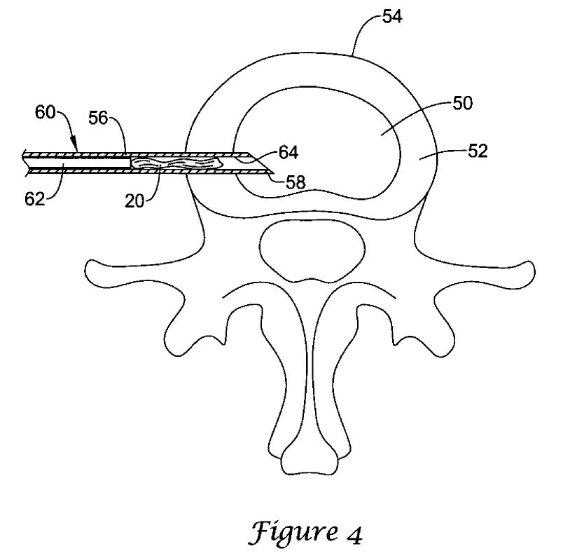

FIGS. 4-9 illustrate one possible procedure of installing the nucleus replacement prosthesis 10 in a nuclear void of an intervertebral disc. During a medical procedure, a desired procedural technique may be performed to access an intervertebral disc 54 of the spinal column. For instance, the intervertebral disc 54 may be accessed through a posterior, anterior, lateral, posterio-lateral approach or other desired procedural technique in an open, percutaneous, or minimally invasive manner. Having gained access to the intervertebral disc, a discectomy may be performed to remove a portion of the intervertebral disc, such as a native nucleus pulposus, creating a nuclear void 50 surrounded by the annulus fibrosus 52 of the intervertebral disc 54. The discectomy may be performed by manipulating one or more tissue removal tools, such as surgical cutters, rongeurs or other tools, through an access device inserted through an incision to the intervertebral disc 54. A discectomy involves the removal of at least a portion of the native nucleus pulposus of the interevertebral disc 54, and often times the entire native nucleus pulposus is removed. In some instances, a small portion of tissue of the annulus fibrosus 52 is also removed, leaving a nuclear void 50 surrounded by the annulus fibrosus 52, or remaining portion of the annulus fibrosus 52.

As shown in FIG. 4, having created a nuclear void in the intervertebral disc 54, a prosthesis insertion tool 56 may be inserted through the annulus fibrosus 52 into the nuclear void 50. The prosthesis insertion tool 56 may include a tubular member 56 having a lumen 64 therein and a sharpened or pointed distal end 58 to facilitate piercing the annulus fibrosus 52 with minimal tissue damage. An elastic porous construct 20 of a nucleus replacement prosthesis 10 may be loaded into the lumen 64 prior to or subsequent insertion of the prosthesis insertion tool 56 into the nuclear void 50. When loaded in the lumen 64 of the prosthesis insertion tool 56, the porous construct 20 may be compressed into a compressed state. In other embodiments, the prosthesis insertion tool 56 may have any desired configuration configured to contain the porous construct 20 in a compressed state for deployment within the nuclear void 50 of the intervertebral disc 54. The prosthesis insertion tool 56 may be utilized to deliver the porous construct 20 into the nuclear void 50 of the intervertebral disc 54 in a compressed state.

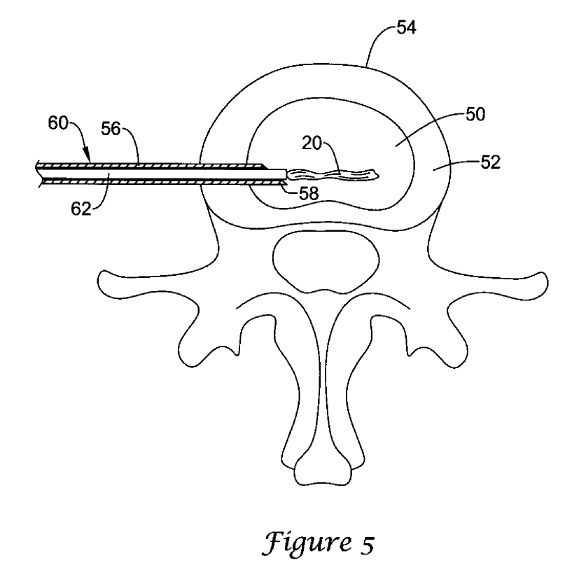

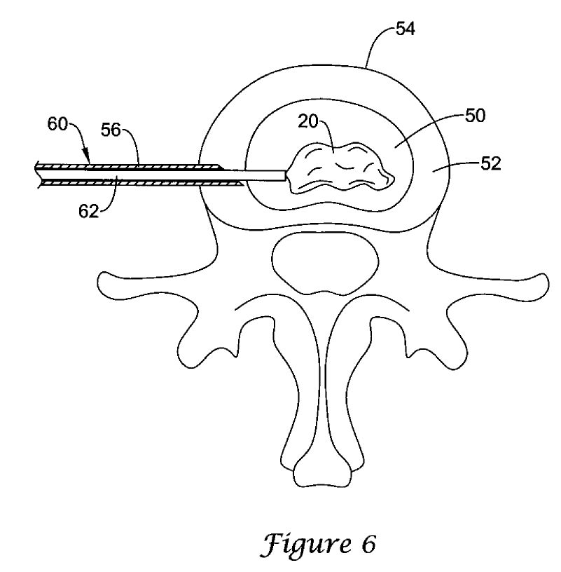

The prosthesis insertion tool 56 may also include an actuation device, illustrated as a pusher member 62, which may be selectively actuated to expel the porous construct 20 out of the lumen 64 of the prosthesis insertion tool 56 at a desired time. As shown in FIG. 5, the pusher member 62 may be actuated distally, expelling the porous construct 20 into the nuclear void 50 from the distal end 58 of the tubular member 56. No longer constrained by the tubular member 56, the porous construct 20 may automatically expand toward its predisposed enlarged or expanded state, as shown in FIG. 6. In other words, as compressive forces applied to the porous construct 20 by the prosthesis insertion tool 56 are removed from the porous construct 20, the porous construct 20 may automatically expand outward toward the annulus fibrosus 52 to assume a biased expanded state. Expansion of the porous construct 20 may be achieved without the need to hydrate the porous construct 20 and/or without further manipulation, alteration, or modification of the porous construct 20.

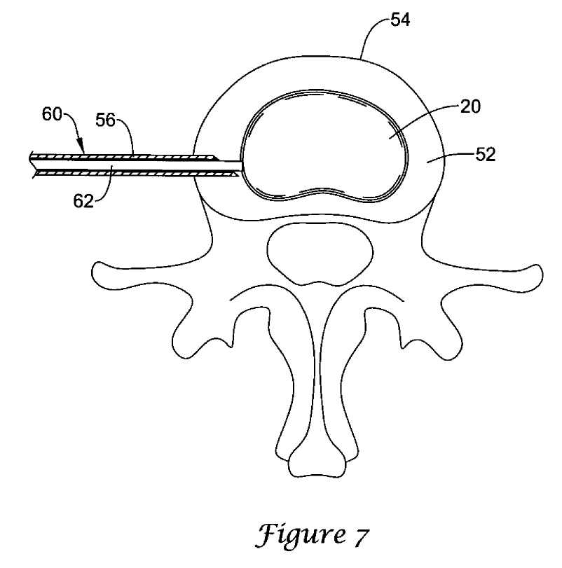

After deployment of the porous construct 20, the prosthesis insertion tool 56 may be withdrawn from the nuclear void 50. Uninhibited expansion of the porous construct 20 may be achieved until the porous construct is expanded to a size and shape which substantially fills the nuclear void 50, at which point the outer extents of the porous construct 20 come into contact with the annulus fibrosus 52 of the intervertebral disc 54, shown in FIG. 7. In some instances the annulus fibrosus 52 may inhibit further expansion of the porous construct 20. In other instances, the containing component 24 may inhibit expansion of the porous construct 20 beyond a desired expanded state.

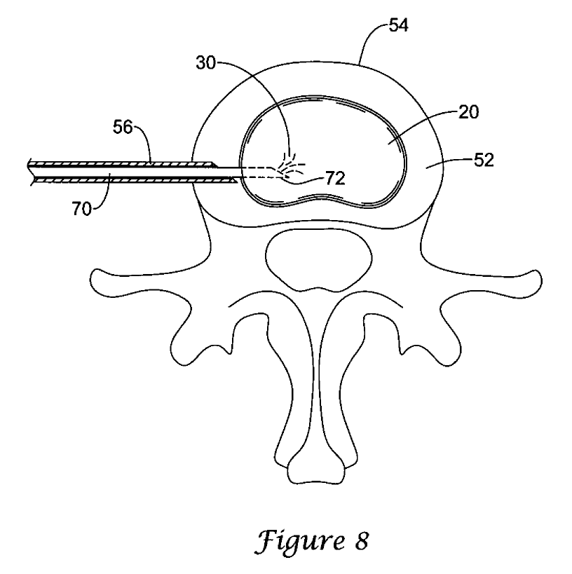

As shown in FIG. 8, once the porous construct 20 has reached its expanded state in the nuclear void 50, an injection tool 70 may be inserted through the annulus fibrosus 52 to the porous construct 20. In some instances, the injection tool 70 may be inserted through the lumen 64 of the tubular member 56, for example, after removal of the pusher member 62 from the lumen 64. In other embodiments, the tubular member 52 of the prosthesis insertion tool 56 may be removed and subsequently the injection tool 70 may be advanced to the porous construct 20 through the annulus fibrosus 52. In some instances, the injection tool 70 may be advanced to the porous construct 20 with the aid of an outer tubular member, access device, cannula and/or guide tube (not shown), or the injection tool 70 may be advanced to the porous construct 20 without the aid of another component. The injection tool 70 may be advanced to the porous construct 20 such that the distal end 72 of the injection 70, which may include a sharpened or pointed tip, is located in contact with the porous construct 20 and/or advanced into the porous construct 20 to an interior portion of the porous construct 20.

A substance 30 may be injected into the pores and/or cavity of the porous construct 20 through the injection tool 70, as shown in FIG. 8. For instance, the substance 30 may be injected through a lumen of the injection tool 70 and out the distal end 72 of the injection tool 70. The substance 30, which may be a fluid, flowable solid, or other injectable material, may occupy the pores of the porous construct 20 and flow between pores of the porous construct 20 to fill or substantially fill the pores of the porous construct 20. Several possible materials which may be injected into the pores of the porous construct 20 have been discussed above. The material of the porous construct 20 may be insufficient to withstand the spinal loads exerted on the nucleus replacement prosthesis 10 without filling or substantially filling the pores of the porous construct 20 with a substance 30. The presence of the substance 30 in the pores of the porous construct 20 may be found to provide the nucleus replacement prosthesis 10 with sufficient strength/support to withstand spinal loads exerted on the nucleus replacement prosthesis 10 along the spinal column.

In some embodiments, the substance 30 occupying the pores 28 may be chosen to provide the nucleus replacement prosthesis 10 with a degree of dynamic loading/offloading capabilities. In some embodiments the substance 30 may post-operatively flow between adjacent interconnected pores of the porous construct 20 during normal activities through cyclic loading/offloading of the nucleus replacement prosthesis 10, resembling the diurnal pumping action associated with a native nucleus pulposus of an intervertebral disc. The movement of the substance 30 into and out of pores of the porous construct 20 may create a poroelastic effect on the construct.

In embodiments in which the substance 30 may be an in situ curing material, the substance 30 may cure after being injected into the pores of the porous construct 20. In some embodiments, the substance 30 may be chosen to be self-hardening or self-curable, or hardenable or curable upon the application of heat, light, air, a curing agent or other hardening or curing means. The substance 30 may be chosen to harden or cure shortly after injection into the pores or over a period of hours or days, in some instances.

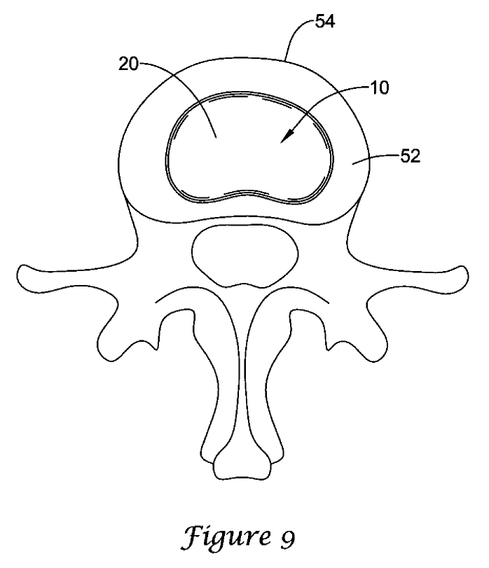

FIG. 9 illustrates the nucleus replacement prosthesis 10 surrounded by the annulus fibrosus 52 of the intervertebral disc 54. The pores and/or cavity of the porous construct 20 has been filled with a substance 30 giving the nucleus replacement prosthesis 10 support capabilities, and thus the nucleus replacement prosthesis 10 is capable of functioning as a native nucleus pulposus, supporting spinal loads transferred along the spinal column through the intervertebral disc 54.

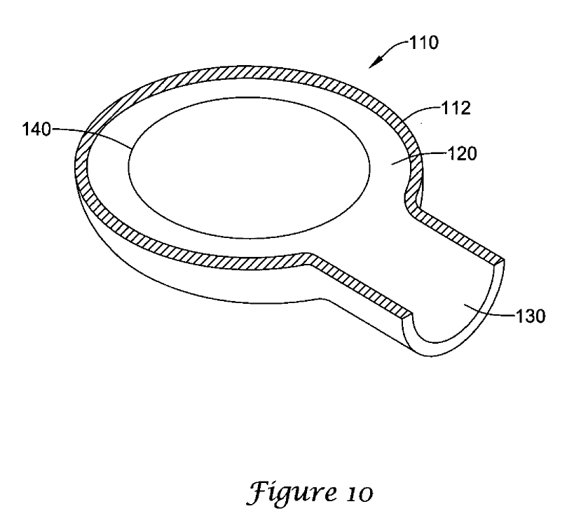

FIG. 10 illustrates another embodiment of a nucleus replacement prosthesis 110. The nucleus replacement prosthesis 10 may include an enclosure or bag 112 similar to the bag disclosed in U.S. Patent Application Publication NO. 2007/0038301, incorporated herein by reference. As shown in FIG. 10, the top portion of the enclosure or bag 112 has been removed to more clearly show the interior of the enclosure or bag 112 and possible contents of the enclosure or bag 112.

The bag 112 may include an interior cavity or chamber 120 into which a construct 140, such as a porous construct or a non-porous construct, may be inserted. In some embodiments the construct 140 may be similar to the porous construct 20 discussed above, whereas in other embodiments the construct 140 may be of another form or configuration. For instance, in some embodiments the construct 140 may be a sinusoidal-shaped structure similar to that disclosed in U.S. Patent Application Publication NO. 2007/0038301. The construct 140 may be inserted into the chamber 120 through an opening 130. The opening 130 may then be closed and/or sealed if desired.

The construct 140 may be formed of any desired material, including those materials discussed above regarding the porous construct 20. In some embodiments, the construct 140 may be formed of a polymer material having surface-modifying endgroups (SME) and/or surface-modifying macromolecules (SMM), creating hydrophilic surfaces on the construct 140 formed of a non-hydrophilic polymer. Thus, in some embodiments the construct 140 may have an affinity for interdiscal fluid, water and/or other fluid, drawing the interdiscal fluid, water and/or other fluid into pores, openings, interstices or cavities of the construct 140. Some surface-modifying endgroups mentioned above, include silicone, sulfonate, fluorocarbon, polyethylene oxide, and hydrocarbon chains. Surface-modifying macromolecules are oligomeric fluoropolymers synthesized by polyurethane chemistry and tailored with fluorinated end groups.

In some embodiments, a substance, such as substance 30 discussed above, which may be a filler material, a fluid, or other material, may be introduced into the pores and/or cavities of the construct 140 and/or within the chamber 120 of the bag 112 around the construct 140. In some embodiments, the substance may contribute to providing the nucleus replacement prosthesis 110 with sufficient structure to carry loads experienced by an intervertebral disc in conjunction with the annulus fibrosus.

Although the prosthesis 10, 110 has been illustrated as a replacement for a native nucleus pulposus of an intervertebral disc, in some instances, the prosthesis 10, 110 may be a total disc prosthesis configured to replace the entire or substantially the entire intervertebral disc of a spinal column including the annulus fibrosus or a substantial portion of the annulus fibrosus.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A nucleus replacement prosthesis for a nucleus of an intervertebral disc, the nucleus replacement prosthesis comprising:
an elastic porous construct formed from a non-hydrogel material, the porous construct having a plurality of interconnected pores, the elastic porous construct being compressible by the application of an applied compressive force from an enlarged state to a smaller compressed state;
wherein the elastic porous construct is predisposed to assume the enlarged state in the absence of the applied compressive force.

2. The nucleus replacement prosthesis of claim 1, further comprising a substance substantially filling the plurality of interconnected pores.

3. The nucleus replacement prosthesis of claim 2, wherein the substance flows between adjacent pores of the elastic porous construct to provide poroelastic behavior.

4. The nucleus replacement prosthesis of claim 3, wherein the substance flowing between adjacent pores of the elastic porous construct resembles diurnal pumping of the nucleus of the vertebral disc.

5. The nucleus replacement prosthesis of claim 1, further comprising a containing component surrounding the elastic porous construct.

6. The nucleus replacement prosthesis of claim 5, wherein the containing component is a non-porous cover.

7. The nucleus replacement prosthesis of claim 5, wherein the containing component is a permeable cover.

8. The nucleus replacement prosthesis of claim 5, wherein the containing component is an outer region of the elastic porous construct having filled pores.

9. The nucleus replacement prosthesis of claim 5, wherein the containing component limits expansion of the elastic porous construct.

10. The nucleus replacement prosthesis of claim 5, wherein a fluid is confined in the interconnected pores of the elastic porous elastic construct by the containing component.

11. The nucleus replacement prosthesis of claim 1, further comprising a curable material substantially filling the plurality of interconnected pores.

12. The nucleus replacement prosthesis of claim 1, wherein the non-hydrogel material includes surface-modifying endgroups.

13. The nucleus replacement prosthesis of claim 1, wherein the elastic porous construct includes an interior cavity.

14. The nucleus replacement prosthesis of claim 13, wherein the interior cavity is substantially larger than the pores of the elastic porous construct.

15. The nucleus replacement prosthesis of claim 14, wherein the cavity contains a fluid.

16. The nucleus replacement prosthesis of claim 15, wherein fluid in the cavity flows into the interconnected pores during spinal loading and fluid flows back into the cavity during spinal offloading.

17. A nucleus replacement prosthesis for a nucleus of an intervertebral disc, the nucleus replacement prosthesis comprising:
an elastic porous construct having a plurality of interconnected pores, the elastic porous construct being compressible by the application of an applied compressive force from an enlarged state to a smaller compressed state;
wherein the elastic porous construct is predisposed to assume the enlarged state in the absence of the applied compressive force without hydration; and
a substance located in the interconnected pores of the elastic porous construct.

18. The nucleus replacement prosthesis of claim 17, wherein hydrogen bonds are not present between the substance and the elastic porous construct.

19. The nucleus replacement prosthesis of claim 17, wherein the elastic porous construct is formed of a non-crosslinked polymer material.

20. The nucleus replacement prosthesis of claim 17, wherein the substance is a fluid flowing between adjacent pores of the elastic porous construct.

21. The nucleus replacement prosthesis of claim 17, wherein the substance is a curable material disposed in the pores of the elastic porous construct.

22. A method of replacing a nucleus of an intervertebral disc, the method comprising:
removing at least a portion of the nucleus to create a nuclear void within an annulus fibrosus of the intervertebral disc;
inserting a porous construct in a compressed state into the nuclear void;
expanding the porous construct within the nuclear void to an expanded state;
injecting a substance into the expanded porous construct such that the substance fills a plurality of interconnected pores of the porous construct.

23. The method of claim 22, wherein the porous construct is formed of a non-hydrogel material.

24. The method of claim 22, wherein in the expanded state, the expanded porous construct substantially fills the nuclear void.

25. The method of claim 22, further comprising:
curing the substance in situ.

26. The method of claim 22, wherein the substance is a fluid flowing between adjacent pores of the porous construct to provide poroelastic behavior.

27. The method of claim 26, wherein fluid flowing between adjacent pores of the porous construct resembles diurnal pumping of the nucleus.
